(19) United States
(12) Patent Application Publication
(10) Pub. No.: US 2023/0210652 A1
(43) Pub. Date: Jul. 6, 2023

(54) Trilobe Inflatable Implants Methods and Systems

(71) Applicant: John Schelter, Centennial, CO (US)

(72) Inventor: John Schelter, Centennial, CO (US)

(21) Appl. No.: 18/092,151

(22) Filed: Dec. 30, 2022

Related U.S. Application Data
(60) Provisional application No. 63/295,806, filed on Dec. 31, 2021.

(51) Int. Cl. A61F 2/08

(52) U.S. Cl. CPC A61F 2/0811; A61F 2002/0817; A61F 2002/0852; A61F 2230/0036; A61F 2250/0003; A61F 2250/0013

(57) ABSTRACT

Embodiments of the application provide methods and systems for treating rotator cuff injuries including but not limited to a trilobe inflatable implant that may be rolled and even inflated. An implant placement tool with a filler to assist in implanting and filling a trilobe implant. A trilobe implant may be held in place with a parts of a shoulder area or may even be secured with a securement.

This application is a U.S. Nonprovisional Application claiming the benefit of and priority to U.S. Provisional Application No. 63/295,806 filed Dec. 31, 2021, hereby incorporated by reference in its entirety herein.

### BACKGROUND OF THE INVENTION

Loss of normal range of motion in the shoulder can be caused by several different issues, but it may frequently be a result of some form of rotator cuff tendinopathy, glenohumeral capsular insufficiency, or even disproportionate adhesive capsulitis. Co-contraction of the rotator cuff tendons during shoulder movement may be required to achieve normal kinematics and avoid pathologic migration of the glenohumeral articulation. Although the rotator cuff consists of four separate tendons, it may be thought that the most significant tendons include the supraspinatus superiorly, the subscapularis anteriorly, and the infraspinatus posteriorly. If rotator cuff tendinopathy or tears exist, the glenohumeral center of rotation may be altered, and the shoulder may be at risk for impingement or chondral injury and subsequent arthritis; moreover, if the rotator cuff has not yet torn, but tendinopathy or insufficiency may be present, then an altered center of rotation can put the rotator cuff at increased risk for additional damage that may ultimately result in an unrepairable massive rotator cuff tear.

During the natural aging process, the rotator cuff may lose its ability to maintain normal center of rotation during a full range of motion. Consequently, systems and methods are needed that may support the humeral head, augmenting the existing rotator cuff, potentially recruiting the deltoid to protect the cartilage, capsular labral complex, and even assist in restoring normal range of motion.

In the past, implants such as the Stryker INSPACE™ balloon may have been designed to replace the subacromial bursa and may be used in patients older than 65 perhaps with massive irreparable rotator cuff tears. However, such past implants can neglect the naturally occuring subcoracoid and subdeltoid bursal space. Without stabilization perhaps from all three vectors surrounding the humeral head throughout its range of motion, these implants may contribute to an abnormal glenohumeral articulation, and may displace from their intended position. This is perhaps why the implant may not be effective in the younger, potentially more active patients with rotator cuff insufficiency. Additional risks with the INSPACE™ balloon may include a propensity to detach from the inserter prior to inflation, positioning, and even complete displacement of the implant. In such cases, the implant is rendered ineffective, the shoulder is at risk for increased arthritic changes, and a surgeon may need to remove the implant and replace it with a new one.

### SUMMARY OF THE INVENTION

The present application includes a variety of aspects, which may be selected in different combinations based upon the particular application or needs to be addressed. In various embodiments, the application may include inflatable implants, perhaps even trilobe inflatable implants to be used in shoulders or other parts of a body.

It is an object of the application to provide an inflatable implant that can be securely placed in a body, such as in a shoulder area.

It is another object of the application to provide an inflatable implant perhaps with three lobes.

It is yet another object of the application to provide an implant placement tool for inserting, inflating, and perhaps even sealing of an inflatable implant.

Naturally, further objects, goals and embodiments of the application are disclosed throughout other areas of the specification, claims, and drawings.

### BRIEF DESCRIPTION OF THE DRAWINGS

FIG. 1 is a top view of a non-limiting example of an inflated trilobe implant in accordance with some embodiments.

FIG. 2 is an anterior view of a non-limiting example of an inflated trilobe implant placed in a shoulder area in accordance with some embodiments.

FIG. 3 is a lateral view of a non-limiting example of an inflated trilobe implant in a shoulder in accordance with some embodiments.

FIG. 4A is a non-limiting example of an inflated trilobe implant with securements in accordance with some embodiments.

FIG. 4B is a non-limiting example of an inflated trilobe implant with securements to parts of a shoulder area in accordance with some embodiments.

FIG. 5 is a non-limiting example of an implant placement tool attached to a rolled inflatable implant in accordance with some embodiments.

FIG. 6 is a non-limiting example of an implant placement tool attached to a rolled inflatable implant with its sheath removed in accordance with some embodiments.

FIG. 7 is a non-limiting example of an implant placement tool inflating an inflatable implant in accordance with some embodiments.

FIG. 8 is a non-limiting example of an implant placement tool positioning an inflated implant in accordance with some embodiments.

FIG. 9 is a non-limiting example of a cork in accordance with some embodiments.

### DETAILED DESCRIPTION OF THE INVENTIONS

It should be understood that embodiments include a variety of aspects, which may be combined in different ways. The following descriptions are provided to list elements and describe some of the embodiments of the application. These elements are listed with initial embodiments; however, it should be understood that they may be combined in any manner and in any number to create additional embodiments. The variously described examples and preferred embodiments should not be construed to limit the embodiments of the application to only the explicitly described systems, techniques, and applications. The specific embodiment or embodiments shown are examples only. The specification should be understood and is intended as supporting broad claims as well as each embodiment, and even claims where other embodiments may be excluded. Importantly, disclosure of merely exemplary embodiments is not meant to limit the breadth of other more encompassing claims that may be made where such may be only one of several methods or embodiments which could be employed in a broader claim or the like. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

Embodiments of the application may provide a method for treating rotator cuff injuries comprising the steps of providing a rolled trilobe inflatable implant (1); providing an implant placement tool (2); attaching said rolled trilobe inflatable implant to said implant placement tool; inserting said rolled trilobe inflatable implant into a shoulder area (3) of a patient (4) with said implant placement tool; inflating said rolled trilobe inflatable implant to create an inflated trilobe implant (5); sealing said inflated trilobe implant to create a sealed inflated trilobe implant; removing said implant placement tool from said sealed inflated trilobe implant; and perhaps even removing said implant placement tool from said shoulder area of said patient.

Other embodiments may provide a rotator cuff injury treatment apparatus comprising a rollable trilobe inflatable implant (1); an implant placement tool (2) configured to insert said rollable trilobe inflatable implant into a shoulder area of a patient and configured to inflate said rollable trilobe inflatable implant; and perhaps even a seal (17) for said rollable trilobe inflatable implant.

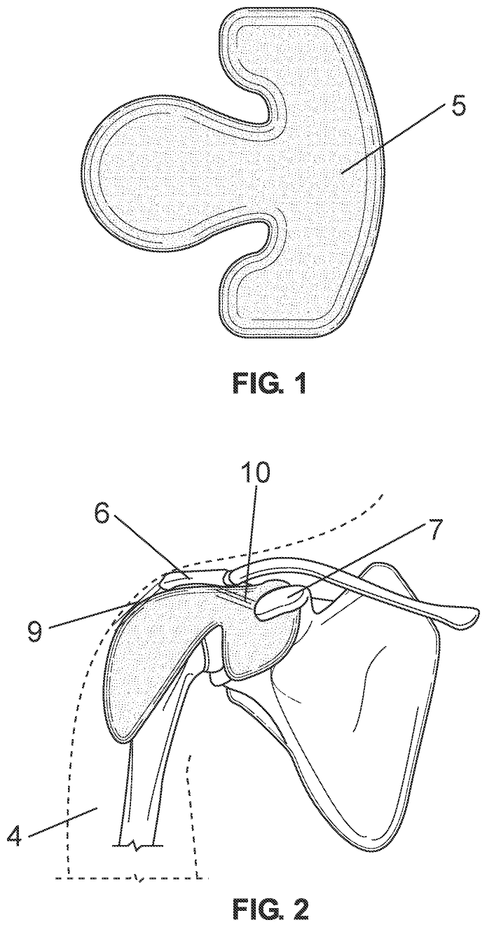

Embodiments of the application may include trilobe inflatable implants which may be used to maintain or even restore the glenohumeral center of rotation to reduce pain, normalize kinematics, and even protect against further impingement, rotator cuff or chondral injury. As may be understood in FIG. 1, a trilobe inflatable implant may have three lobes. An inflated trilobe implant (5) when inserted into a shoulder area may stabilize the humeral head superiorly, anteriorly, laterally, and even posteriorly throughout the entire range of motion of the shoulder.

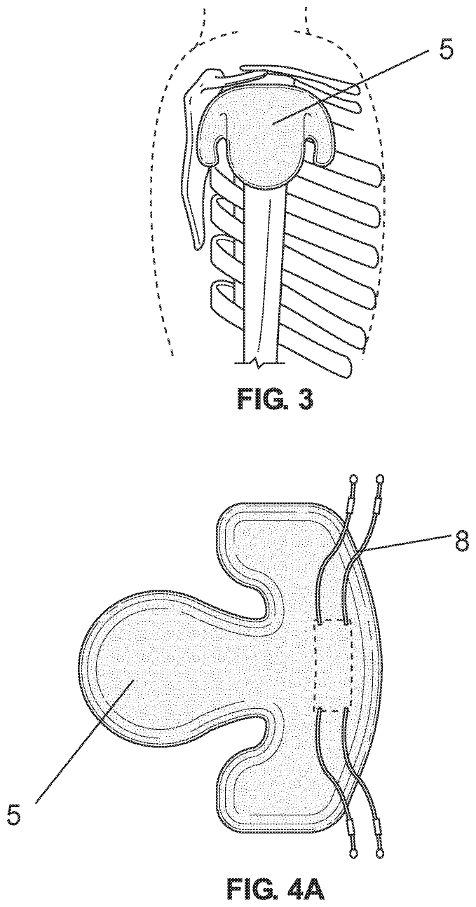

In embodiments and as may be understood in FIG. 2, a trilobe inflatable implant may be inserted and inflated from a superolateral portal with an implant placement tool (2) that may be secured to the implant perhaps throughout the positioning, inflating, and even sealing process. A fully inflated implant may be used to restore the center of rotation and even stabilize the humeral head perhaps by creating a restraint superiorly, anteriorly, posteriorly, and even laterally perhaps as shown in FIG. 3. This restraint and even maintenance of the center of rotation throughout the full range of motion may normally be the function of the four tendons of a healthy rotator cuff.

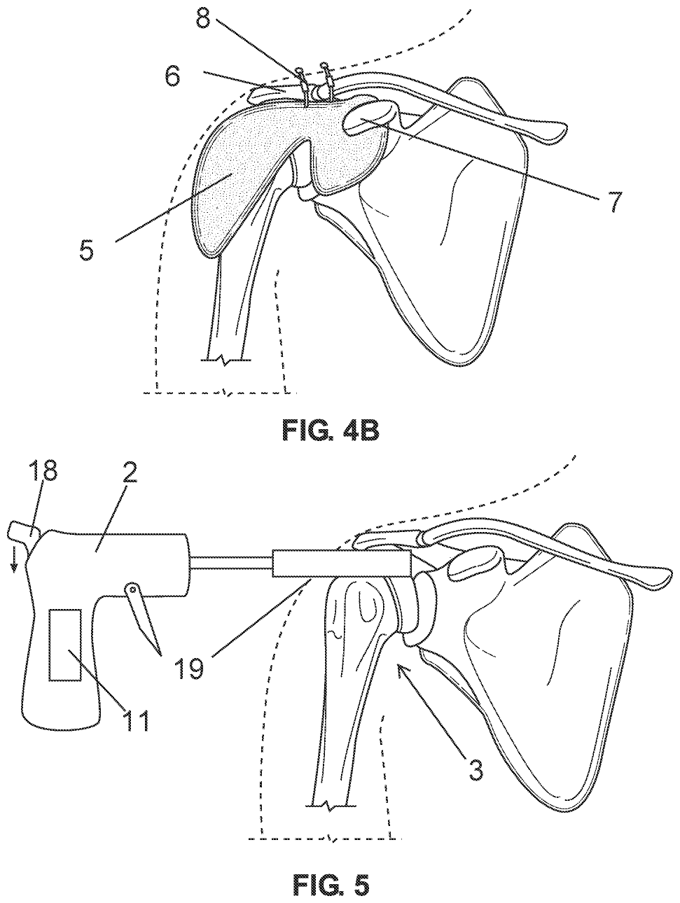

In embodiments such as shown in FIGS. 2, 4A and 4B, a trilobe implant may be secured to the acromion (6) and potentially the coracoid process (7) such as with a securement (8) which may ensure that the position is maintained. A securement (8) may be an adjustable loop tensioning sutures, tapes, bands, any combination thereof, or the like. A trilobe nature of an implant may be held in place by the peripheral structures such as the acromion (6), deltoid (9), coracoacromial ligament (10), coracoid process (7), glenoid, humerus, any combination thereof, or the like, perhaps without any need for securing the implant with a securement. In some embodiments, each of three lobes of an inflated trilobe implant may be held by or even secured to different parts of a shoulder area which can include but is not limited to acromion, deltoid, coracoacromial ligament, glenoid, humerus, coracoid, any combination thereof, or the like.

Past implants such do not have an accurate method for determining inflation size, and subsequent over/under inflation may occur resulting in displacement or loss of efficacy. In embodiments, the application may provide an implant placement tool (2) perhaps with a preloaded and even predetermined amount of filler (11) which may include but is not limited to saline, biologics, antibiotics, inflatable liquid, paste, gas, any combination thereof, or the like to facilitate the inflation phase and even decrease intraoperative errors of over or under inflation.

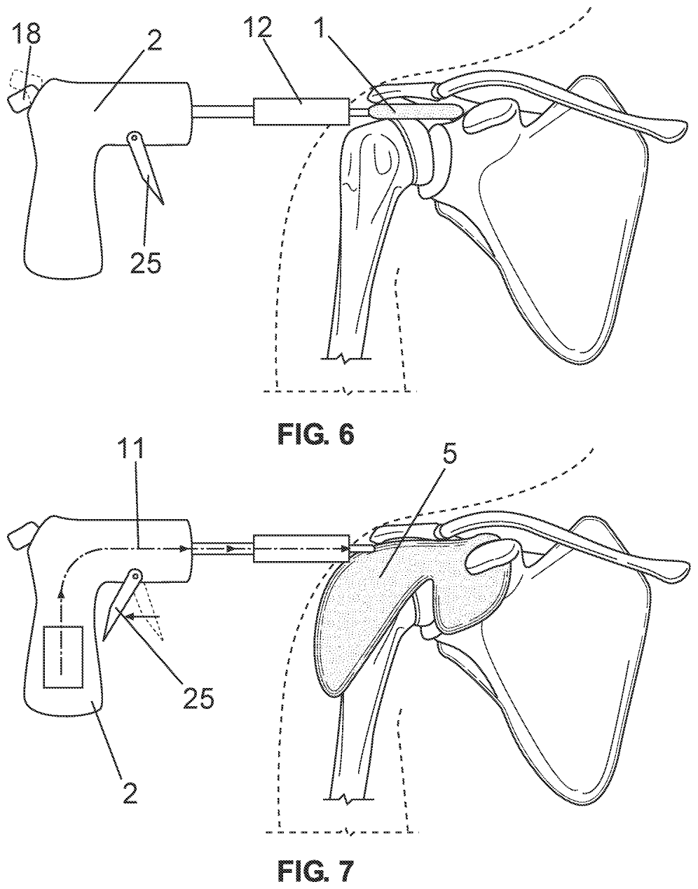

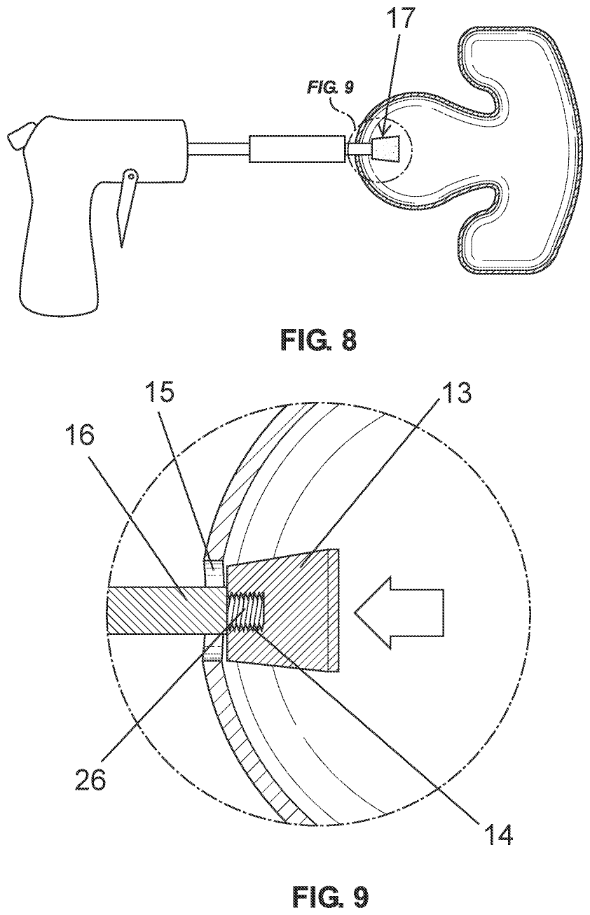

In embodiments, a patient (4) may be sized and an appropriate size implant may be selected, an implant placement tool may be inserted through a lateral portal (19) such as shown in FIG. 5. An implant placement tool, which may be a pistol shaped like tool, may contain a predetermined amount of saline or other inflatable liquid, paste, gas (e.g., nonflammable) or the like perhaps along with a sheath (12) such as to protect a rolled trilobe implant. A sheath (12) may be located over a rolled trilobe inflatable implant. In alternative embodiments, an implant and implant placement tool attachment may be reversed so that insertion could occur through a Neviaser portal perhaps if it is determined that this may be a better way to achieve a more lateral position for the implant while observing range of motion. A superior lever (18) of an implant placement tool may be “cocked” inferiorly perhaps to retract the sheath and expose the rolled implant such as shown in FIG. 6. A trigger (25) may be pulled to a first click perhaps to unroll and even inflate an implant perhaps with a predetermined amount of saline such as shown in FIG. 7. In some situations, a surgeon may inflate or even deflate the implant as needed perhaps to position the humerus, restore the center of rotation, and even reposition the implant. In some embodiments, an additional luer lock may be available on the superior lever of the device. At this point, the implant could be fully inflated. The inflated trilobe implant (5) may then be sealed perhaps with a seal (17) and perhaps by pulling a trigger to a second click such as to plug an aperture (15) with cork (13) as shown in FIG. 8. A tip (16) of the implant placement tool may be threaded (26) into a cork (13) perhaps so that the implant will not fall off before sealing and even positioning has been finalized such as shown in FIG. 9. After the implant has been sealed with the cork, it may still be repositioned. Once a final position may be determined, a final locking of the cork perhaps with its distal threads (14) into an aperture (15) may be achieved. A shoulder may then be taken through a range of motion perhaps to confirm that the center of rotation has been restored, and the inserter may be removed perhaps by rotating the gun counterclockwise until its tip (16) unthreads from the cork.

An implant may be made of a bioabsorbable material perhaps for a single operation. An implant may be made of a non-absorbable material. A rate of absorption and response to physical therapy may be different for each patient. Some embodiments may provide a two-phase procedure where a non-absorbable implant may be used first and a bioabsorbable implant may be used second. A surgeon and physical therapy team may monitor the re-education of the residual rotator cuff and deltoid and restoration of kinematics until they believe that the patient is ready to move into the bioabsorbable phase.

As can be easily understood from the foregoing, the basic concepts of the various embodiments of the present invention(s) may be embodied in a variety of ways. It involves both implant techniques as well as devices to accomplish the appropriate implant. In this application, the implant techniques are disclosed as part of the results shown to be achieved by the various devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the various embodiments of the invention(s) and may not explicitly show how each feature or element can actually be representative of a broader function or of a great variety of alternative or equivalent elements. As one example, terms of degree, terms of approximation, and/or relative terms may be used. These may include terms such as the words: substantially, about, only, and the like. These words and types of words are to be understood in a dictionary sense as terms that encompass an ample or considerable amount, quantity, size, etc. as well as terms that encompass largely but not wholly that which is specified. Further, for this application if or when used, terms of degree, terms of approximation, and/or relative terms should be understood as also encompassing more precise and even quantitative values that include various levels of precision and the possibility of claims that address a number of quantitative options and alternatives. For example, to the extent ultimately used, the existence or non-existence of a substance or condition in a particular input, output, or at a particular stage can be specified as substantially only x or substantially free of x, as a value of about x, or such other similar language. Using percentage values as one example, these types of terms should be understood as encompassing the options of percentage values that include 99.5%, 99%, 97%, 95%, 92% or even 90% of the specified value or relative condition; correspondingly for values at the other end of the spectrum (e.g., substantially free of x, these should be understood as encompassing the options of percentage values that include not more than 0.5%, 1%, 3%, 5%, 8% or even 10% of the specified value or relative condition, all whether by volume or by weight as either may be specified). In context, these should be understood by a person of ordinary skill as being disclosed and included whether in an absolute value sense or in valuing one set of or substance as compared to the value of a second set of or substance. Again, these are implicitly included in this disclosure and should (and, it is believed, would) be understood to a person of ordinary skill in this field. Where the application is described in device-oriented terminology, each element of the device implicitly performs a function. Apparatus claims may not only be included for the device described, but also method or process claims may be included to address the functions of the embodiments and that each element performs. Neither the description nor the terminology is intended to limit the scope of the claims that will be included in any subsequent patent application.

It should also be understood that a variety of changes may be made without departing from the essence of the various embodiments of the invention(s). Such changes are also implicitly included in the description. They still fall within the scope of the various embodiments of the invention(s). A broad disclosure encompassing the explicit embodiment(s) shown, the great variety of implicit alternative embodiments, and the broad methods or processes and the like are encompassed by this disclosure and may be relied upon when drafting the claims for any subsequent patent application. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of embodiments of the invention(s) both independently and as an overall system.

Further, each of the various elements of the embodiments of the invention(s) and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the various embodiments of the invention(s), the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which embodiments of the invention(s) is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of a “insert” should be understood to encompass disclosure of the act of “inserting”—whether explicitly discussed or not—and, conversely, were there effectively disclosure of the act of “inserting”, such a disclosure should be understood to encompass disclosure of an “insert” and even a “means for inserting.” Such changes and alternative terms are to be understood to be explicitly included in the description. Further, each such means (whether explicitly so described or not) should be understood as encompassing all elements that can perform the given function, and all descriptions of elements that perform a described function should be understood as a non-limiting example of means for performing that function. As other non-limiting examples, it should be understood that claim elements can also be expressed as any of: components, programming, subroutines, logic, or elements that are configured to, or configured and arranged to, provide or even achieve a particular result, use, purpose, situation, function, or operation, or as components that are capable of achieving a particular activity, result, use, purpose, situation, function, or operation. All should be understood as within the scope of this disclosure and written description.

Any patents, publications, or other references mentioned in this application for patent are hereby incorporated by reference. Any priority case(s) claimed by this application is hereby appended and hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference. Finally, all references listed in the information statement filed with the application are hereby appended and hereby incorporated by reference, however, as to each of the above, to the extent that such information or statements incorporated by reference might be considered inconsistent with the patenting of the various embodiments of invention(s) such statements are expressly not to be considered as made by the applicant(s).

Thus, the applicant(s) should be understood to have support to claim and make claims to embodiments including at least: i) each of the implant devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such processes, methods, systems or components, ix) each system, method, and element shown or described as now applied to any specific field or devices mentioned, x) methods and apparatuses substantially as described hereinbefore and with reference to any of the accompanying examples, xi) an apparatus for performing the methods described herein comprising means for performing the steps, xii) the various combinations and permutations of each of the elements disclosed, xiii) each potentially dependent claim or concept as a dependency on each and every one of the independent claims or concepts presented, and xiv) all inventions described herein.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrases “comprising”, “including”, “containing”, “characterized by” and “having” are used to maintain the “open-end” claims herein, according to traditional claim interpretation including that discussed in MPEP § 2111.03. Thus, unless the context requires otherwise, it should be understood that the terms “comprise” or variations such as “comprises” or “comprising”, “include” or variations such as “includes” or “including”, “contain” or variations such as “contains” and “containing”, “characterized by” or variations such as “characterizing by”, “have” or variations such as “has” or “having”, are intended to imply the inclusion of a stated element or step or group of elements or steps but not the exclusion of any other element or step or group of elements or steps. Such terms should be interpreted in their most expansive form so as to afford the applicant the broadest coverage legally permissible. The use of the phrase, “or any other claim” is used to provide support for any claim to be dependent on any other claim, such as another dependent claim, another independent claim, a previously listed claim, a subsequently listed claim, and the like. As one clarifying example, if a claim were dependent “on claim 9 or any other claim” or the like, it could be re-drafted as dependent on claim 1, claim 8, or even claim 11 (if such were to exist) if desired and still fall with the disclosure. It should be understood that this phrase also provides support for any combination of elements in the claims and even incorporates any desired proper antecedent basis for certain claim combinations such as with combinations of method, apparatus, process, and the like claims.

Finally, any claims set forth at any time are hereby incorporated by reference as part of this description of the various embodiments of the application, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

What is claimed is:

1. A method for treating rotator cuff injuries comprising the steps of:
providing a rolled trilobe inflatable implant;
providing an implant placement tool;
attaching said rolled trilobe inflatable implant to said implant placement tool;
inserting said rolled trilobe inflatable implant into a shoulder area of a patient with said implant placement tool;
inflating said rolled trilobe inflatable implant to create an inflated trilobe implant;
sealing said inflated trilobe implant to create a sealed inflated trilobe implant;
removing said implant placement tool from said sealed inflated trilobe implant; and
removing said implant placement tool from said shoulder area of said patient.

2. The method as described in claim 1 and further comprising a step of securing part of said rolled trilobe inflatable implant to an acromion and a coracoid process of said shoulder area with a securement.

3. The method as described in claim 2 wherein said securement is chosen from adjustable loop tensioning sutures, tapes, bands, and any combination thereof.

4. The method as described in claim 1 and further comprising a step of holding said inflated trilobe implant in place with a shoulder area chosen from acromion, deltoid, coracoacromial ligament, coracoid, glenoid, humerus, and any combination thereof.

5. The method as described in claim 4 and further comprising a step of securing each of three lobes of said inflated trilobe implant with different parts of said shoulder area chosen from acromion, deltoid, coracoacromial ligament, glenoid, humerus, and coracoid.

6. The method as described in claim 1 wherein said step of inflating said rolled trilobe inflatable implant to create said inflated trilobe implant comprises a step of inflating said rolled trilobe inflatable implant with a predetermined amount of a filler located in said implant placement tool.

7. The method as described in claim 6 wherein said filler comprises a substance chosen from saline, biologics, antibiotics, inflatable liquid, paste, gas, and any combination thereof.

8. The method as described in claim 1 and further comprising a step of deflating said inflated trilobe implant.

9. The method as described in claim 1 and further comprising a step of providing a sheath over said rolled trilobe inflatable implant prior to being inserted in said shoulder area of said patient.

10. The method as described in claim 9 and further comprising a step of removing said sheath from said rolled trilobe inflatable implant with said implant placement tool after said step of inserting said rolled trilobe inflatable implant into said shoulder area of said patient.

11. The method as described in claim 1 and further comprising a step of repositioning said sealed inflated trilobe implant.

12. The method as described in claim 1 wherein said step of sealing said inflated trilobe implant comprises a step of sealing said inflated trilobe implant with a cork.

13. The method as described in claim 12 and further comprising a step of final locking said sealed trilobe implant by securing distal threads of said cork into an aperture of said trilobe implant.

14. The method as described in claim 13 wherein said step of removing said implant placement tool from said sealed inflated trilobe implant comprises a step of rotating said implant placement tool until a tip unthreads from said cork.

15. The method as described in claim 1 wherein said rolled trilobe inflatable implant is made of a material chosen from bioabsorbable material and a non-absorbable material.

16. The method as described in claim 6 and further comprising a step of sizing said shoulder area of said patient and determining an amount of said filler to be inserted into said rolled trilobe inflatable implant.

17. A rotator cuff injury treatment apparatus comprising:
a rollable trilobe inflatable implant;
an implant placement tool configured to insert said rollable trilobe inflatable implant into a shoulder area of a patient and configured to inflate said rollable trilobe inflatable implant; and
a seal for said rollable trilobe inflatable implant.
